(19) United States
(12) Patent Application Publication
(10) Pub. No.: US 2009/0176207 A1
(43) Pub. Date: Jul. 9, 2009

(54) Methods for Determining Risk of Developing Regular Smoking Behavior

(75) Inventors: Rosalind Neuman, St. Louis, MO (US); Richard D. Todd, St. Louis, MO (US)

Correspondence Address: WASHINGTON UNIVERSITY-SNR;C/O SONNENSCHEIN NATH & ROSENTHAL L.L.P, P.O. BOX 061080, WACKER DRIVE STATION , SEARS TOWER, CHICAGO, IL, 60606, US

(73) Assignee: Washington University, St. Louis, MO (US)

(21) Appl. No.: 11/571,245

(22) PCT Filed: Jun. 23, 2005
(86) PCT No.: PCT/US2005/022779

§ 371 Date: Aug. 11, 2008

Related U.S. Application Data
(60) Provisional application No. 60/582,159, filed on Jun. 23, 2004.

(51) Int. Cl. C12Q 1/68; C40B 40/08

(52) U.S. Cl. 435/6; 506/17

(57) ABSTRACT

The present invention relates in general to human genetic polymorphisms and their association with human health and to methods and materials for analyzing allelic variations, and to the use of genetic polymorphisms in the diagnosis and treatment of smoking behavior and nicotine dependence. Provided herein are methods for determining risk in a subject of developing regular smoking behavior. Also provided are primers, probes, microarrays, and kits related thereto.

### CROSS-REFERENCE TO RELATED APPLICATIONS

This application claims priority to Provisional Application U.S. Ser. No. 60/582,159, filed Jun. 23, 2004.

### STATEMENT REGARDING FEDERALLY SPONSORED RESEARCH OR DEVELOPMENT

Research relating to the invention was supported in part by NIMH grant R01 MH52813, NINDS grant R01 NS 43762 and NIAAA grant R01 AA 13640. The Government has certain rights in the invention.

### REFERENCE TO A SEQUENCE LISTING

The Sequence Listing, which is a part of the present disclosure, includes a text file comprising nucleotide and/or amino acid sequences of the present invention on a floppy disk. The subject matter of the Sequence Listing is incorporated herein by reference in its entirety.

### BACKGROUND OF THE INVENTION

1. Field of the Invention

The present invention relates in general to human genetic polymorphisms and their association with human health, to methods and materials for analyzing allelic variations, and to the use of genetic polymorphisms in the diagnosis and treatment of smoking behavior and nicotine dependence.

2. Description of the Related Art

The health consequences of smoking represent a major source of morbidity, mortality and economic loss worldwide. Though there is little doubt that a variety of factors contribute to the development of smoking problems such as availability, use by peers, initial reaction to smoking, etc., regular smoking and/or nicotine dependence are moderately heritable behaviors.

The psychological effects of nicotine are thought to be due to stimulation of neural nicotine acetylcholine receptors. Nicotinic acetylcholine receptors are pentameric protein complexes which serve as ligand gated ion channels (see, e.g., Hogg R C, Raggenbass M, Bertrand D (2003), Nicotinic acetylcholine receptors: from structure to brain function. Reviews of Physiology Biochemistry & Pharmacology. 147: 1-46). Individual receptor subunits are coded for by at least a dozen genes, many of which have been investigated using both knock-out and knock-in molecular approaches (see, e.g., Champtiaux N, Changeux J P (2004), Knockout and knockin mice to investigate the role of nicotinic receptors in the central nervous system. Progress in Brain Research. 145: 235-51). The two major brain receptor complexes are an α4β2 heteropentamer with high affinity for nicotine and an α7 homopentamer with low affinity for nicotine. In hippocampus, distinct functional nicotinic receptor subtypes modulate GABAergic and glutaminergic inputs (see, e.g., Alkondon M, Albuquerque EX (2004), The nicotinic acetylcholine receptor subtypes and their function in the hippocampus and cerebral cortex. Progress in Brain Research. 145: 109-20). In the cerebral cortex, layer V pyramidal neuron inhibition is enhanced by activation of high affinity α4 β2 receptor complexes and possibly by low affinity α7 complexes. In the basal ganglia, there is dense intermingling of both dopaminergic and cholinergic systems involving nicotinic receptors. In particular, there is dense cholinergic innervation of striatal dopaminergic neurons via high affinity nicotinic receptors on local interneurons (Zhou F M, Wilson C, Dani J A (2003), Muscarinic and nicotinic cholinergic mechanisms in the mesostriatal dopamine systems. Neuroscientist. 9: 23-36).

The function of individual neural nicotinic acetylcholine receptor subunits, with respect to the behavioral effects of nicotine, have been explored recently in several animal studies. For example, it has been known for some time that nicotine at levels experienced by smokers enhances dopamine release (Grady S, Marks M J, Wonnacott S, Collins A C (1992) Characterization of nicotinic receptor-mediated [3H]dopamine release from synaptosomes prepared from mouse striatum. Journal of Neurochemistry. 59(3):848-56). Mice lacking the β2 nicotine receptor subunit are deficient in this response (F M Zhou et al. (2001), Endogenous nicotinic cholinergic activity regulates dopamine release in the striatum. Nature Neuroscience. 4: 1224-9). Similarly, analyses of crosses of mice strains with both gain of function and loss of function mutations demonstrate that the α4 β2 neuronicotinic acetylcholine receptor complex modulates nicotine effects on the acoustic startle response (J C Owens et al. (2003), Alpha4beta2* Nicotinic Acetylcholine Receptors Modulate the Effects of Ethanol and Nicotine on the Acoustic Startle Response. Alcoholism: Clinical and Experimental Research 27: 1867-1875). In a variety of behavioral tests such as nicotine self-administration and nicotine-induced behavioral sensitization in rats, α4 β2 partial agonists reduce nicotine dependence (C. Cohen et al. (2003), SSR591813, a novel selective and partial alpha4beta2 nicotinic receptor agonist with potential as an aid to smoking cessation. Journal of Pharmacology & Experimental Therapeutics. 306: 407-20). Finally, studies using the stroke-prone spontaneously hypertensive rat as an animal model of attention deficit hyperactivity disorder (ADHD) showed that nicotine improves spontaneous alternative deficit behaviors, and an α4 β2 nicotinic acetylcholine receptor antagonist blocked the nicotine-induced improvement of spontaneous behavior, whereas α7 antagonists did not (K. Ueno et al. (2002), Alpha4beta2 nicotinic acetylcholine receptor activation ameliorates impairment of spontaneous alternation behavior in stroke-prone spontaneously hypertensive rats, an animal model of attention deficit hyperactivity disorder. Journal of Pharmacology & Experimental Therapeutics. 302: 95-100).

Despite significant animal evidence for the involvement of the α4 β2 neural nicotinic receptor complex in the positive effects of nicotine as well as nicotine dependence, the roles of these high affinity nicotine receptor subunits in human smoking have been little investigated, and what little is known has not been very helpful. For example, studies of the association of β2 neural nicotinic acetylcholine receptor gene (CHRNB2) polymorphisms with history of smoking and nicotine dependence have found no evidence for association (see, e.g. Silverman M A, Neale M C, Sullivan P F, Harris-Kerr C, Wormley B, Sadek H, Ma Y, Kendler K S, Straub RE (2000), Haplotypes of four novel single nucleotide polymorphisms in the nicotinic acetylcholine receptor beta2-subunit (CHRNB2) gene show no association with smoking initiation or nicotine dependence. American Journal of Medical Genetics 96: 646-53).

A need therefore remains for a better understanding of cellular and molecular mechanisms underlying smoking behavior, so that better treatments and prevention strategies can be investigated and developed. A need also remains for improved methods of identifying individuals at relatively higher risk of developing regular smoking behavior, so that early intervention can be instituted.

### SUMMARY OF THE INVENTION

Briefly, therefore, methods and materials are provided for determining risk in an individual of developing regular smoking behavior.

In one embodiment of the invention, there is provided a method for determining risk in an individual of developing regular smoking behavior comprising, in a biological sample from the subject, analyzing a polynucleotide sequence to detect the presence or absence of an allelic variant of a polymorphic region of exon/intron 5 region of CHRNA4. In one embodiment of the method, the polymorphic region comprises the sequence of SEQ ID NO: 1. In another embodiment of the method, the allelic variant of the polymorphic region comprises at least one single nucleotide polymorphism (SNP), which is selected from the snp 1 T and snp 2 A alleles. Alternatively, the allelic comprises two single nucleotide polymorphisms (SNPs) consisting of the snp 1 T and snp 2 A alleles.

In another embodiment of the invention, there is provided a method for determining whether a subject is at risk for developing regular smoking behavior, the method comprising, in a biological sample from the subject, analyzing a polynucleotide sequence to detect the presence or absence of at least one DNA marker in exon/intron 5 region of CHRNA4, wherein the DNA marker is associated with increased risk of developing regular smoking behavior. In one embodiment of the method, the at least one DNA marker comprises at least one SNP selected from the snp 1 T and snp 2 A alleles. In another embodiment of the method, the at least one DNA marker comprises two SNPs consisting of the snp 1 T and sup 2 A alleles.

In another embodiment of the invention, there is provided a method for detecting the presence or absence in a subject of at least one allelic variant that is associated with increased risk of developing regular smoking behavior, the method comprising detecting the presence or absence of at least one SNP in exon/intron 5 region of CHRNA4, wherein the presence of at least one SNP is associated with increased risk of developing regular smoking behavior. In one embodiment of the method, detecting the presence or absence of at least one SNP comprises detecting the presence or absence of at least one SNP selected from the snp 1 T and snp 2 A alleles. In another embodiment of the method, detecting the presence or absence of at least one SNP comprises detecting the presence or absence of two SNPs consisting of the snp 1 T and snp 2 A alleles.

In another embodiment of the invention, there is provided a method for detecting in a subject a predisposition to developing regular smoking behavior, comprising, on a CHRNA4 gene obtained from the subject, detecting the presence or absence of an allelic variant of a polymorphic region of exon/intron 5 that is associated with increased risk of developing regular smoking behavior, wherein the polymorphic region comprises SEQ ID NO: 1, and wherein the presence of the allelic variant in the subject is indicative of a predisposition to developing regular smoking behavior in the subject as compared to a subject in which the allelic variant is not present. In one embodiment of the method, the allelic variant comprises a SNP selected from the snp 1 T and snp2A alleles. In another embodiment of the method, the allelic variant comprises two SNPs consisting of the snp 1 T and snp2A alleles. In yet another embodiment of the method, detecting the presence or absence of the allelic variant comprises a method selected from the group consisting of: allele specific hybridization, primer specific extension, oligonucleotide ligation assay, restriction enzyme site analysis and single-stranded conformation polymorphism analysis.

In another embodiment of the invention, there is provided a primer or probe that specifically hybridizes adjacent to or at a polymorphic region of CHRNA4 gene comprising SEQ ID NO: 1, wherein allelic variants of the polymorphic region are associated with increased risk of developing regular smoking. In one embodiment of the primer or probe, the polymorphic region comprises an allelic variant comprising a SNP selected from the snp1T and snp2A alleles. In another embodiment of the primer or probe, the polymorphic region comprises an allelic variant comprising two SNPs consisting of the snp1T and snp2A alleles.

In another embodiment of the invention, there is provided a kit for indicating whether a subject has an increased risk of developing regular smoking behavior, the kit comprising: at least one probe or primer that specifically hybridizes adjacent to or at a polymorphic region of CHRNA4 comprising SEQ ID NO: 1, wherein allelic variants of the polymorphic region are associated with increased risk of developing regular smoking; and instructions for use of the kit for indicating whether the subject has increased risk of developing regular smoking behavior. In one embodiment of the kit, the polymorphic region comprises an allelic variant comprising a SNP selected from the snp1T and snp2A alleles. In another embodiment of the kit, the polymorphic region comprises an allelic variant comprising two SNPs consisting of the snp1T and snp2A alleles.

In another embodiment of the invention, there is provided a microarray, comprising a nucleic acid having a sequence of a polymorphic region of CHRNA4 comprising SEQ ID NO: 1, and that is associated with increased risk of developing regular smoking behavior. In one embodiment of the microarray, the polymorphic region comprises an allelic variant comprising a SNP selected from the snp1T and snp2A alleles. In another embodiment of the microarray, the polymorphic region comprises an allelic variant comprising two SNPs consisting of the snp1T and snp2A alleles.

### BRIEF DESCRIPTION OF THE DRAWINGS

FIG. 1 is a bar graph illustrating comparative odds ratios for occurrence of predictors of regular smoking in individuals diagnosed as Latent Class Defined Attention deficit/hyperactivity disorder (ADHD);

FIG. 2a is a graph illustrating Kaplan-Meir Survival estimates for age of first drink for individuals with Inattentive ADHD;

FIG. 2b is a graph illustrating Kaplan-Meir Survival estimates for age of first cigarette for individuals with Inattentive ADHD; and

FIG. 3 shows the DNA sequence and locations of polymorphisms in the Exon/Intron 5 region of the CHRNA4 gene.

### DETAILED DESCRIPTION OF THE PREFERRED EMBODIMENTS

### Abbreviations and Definitions

“Allele”: As used herein, the term “allele” refers to any of the two or more alternative forms of a gene that occur at the same locus on a chromosome.

“Allelic variant”: As used herein, the term “allelic variant” refers to an alternative form of CHRNA4 which includes one or more SNP's, the occurrence of which is associated with increased risk of developing smoking behavior in a subject carrying one (heterozygous) or two (homozygous) copies of the alternative form.

“Associated”: As used herein in connection with the relationship between certain alleles and increased risk of developing regular smoking behavior, the terms “associated” and “association” refer to the co-occurrence of certain CHRNA4 alleles and regular smoking behavior in subjects, and also to the co-occurrence of the CHRNA4 alleles and the inattentive form of attention deficit hyperactivity disorder (ADHD) in subjects.

“Association”: As used herein in connection with the relationship between certain alleles and increased risk of developing regular smoking behavior, the term “association” refers to the co-occurrence in a subject of the one or both of the snp 1 T and snp 2 A alleles and increased risk of developing regular smoking behavior, as established using association analysis as described herein.

“CHRNA4”: As used herein, the term “CHRNA4” refers to the human high affinity nicotinic acetylcholine receptor subunit gene, and alleles thereof (National Center for Biotechnology Information (NCBI) RefSeq No: NM_{—}000744; GenBank accession no: L35901).

“SNP”: As used herein, the term “SNP” refers to a single nucleotide polymorphism, which is a variation in a genomic DNA sequence that consists of an alteration of a single “base”, or nucleotide (A, T, C, or G) in the nucleotide sequence.

“snp 1 T”: As used herein, the term “snp 1 T” refers to the substitution of “T” for “C” at amino acid position 543 of the CHRNA4 protein sequence.

“snp 2 A”: As used herein, the term “snp 2 A” refers to the substitution of “A” for “G” at amino acid position 553 of the CHRNA4 protein sequence.

“Subject”: As used herein, the term “subject” refers to both a human having or suspected of having an increased risk of developing regular smoking behavior, and an asymptomatic human who may be tested for increased risk of developing regular smoking behavior. At each base pair position on a gene the human may be homozygous for an allele or the human may be a heterozygote.

“Risk”: As used herein, the term “risk” refers to the possibility of being subject to a hazard, namely of developing regular smoking behavior.

“Increased risk”: As used herein, the term “increased risk” refers to the characteristic of a subject having a predisposition to developing regular smoking behavior in the sense that the subject has a greater likelihood of developing such behavior than the general population.

“Polymorphic region”: As used herein with respect to CHRNA4, the term “polymorphic region” refers to a DNA sequence having the nucleotide sequence of SEQ ID NO: 1, corresponding to the Exon/Intron 5 region of CHRNA4, which is known to vary in the general population by certain known SNP's.

“Regular smoking behavior”: As used herein, the term “regular smoking behavior” refers to consistent, daily or almost daily use of one or more cigarettes for a minimum period of three weeks, as self-reported by subjects.

Methods and materials for determining risk of developing regular smoking behavior are based in part on the discovery that two commonly occurring DNA markers from the high affinity nicotinic acetylcholine receptor subunit gene CHRNA4 are associated with an increased risk of progression to regular smoking behavior after initial exposure to smoking. More specifically, increased risk of developing regular smoking behavior is associated with the occurrence in a subject of one or both of the following two single nucleotide polymorphisms (SNP's) in exon 5 of the CHRNA4 gene: the first (“snp 1 T”), is located at amino acid position 543 of the CHRNA4 protein sequence, is identified as rs1044396 mapped to human chromosome 20, as described in the NCBI database. The second (“snp 2 A”), is located at amino acid position 553 of the CHRNA4 protein sequence, identified as rs1044397 mapped to human chromosome 20, as described in the NCBI database.

Current pharmaceutical and behavioral approaches used to change smoking behavior have undesirable side effects and are not highly effective. The identification of therapeutic alternatives is greatly facilitated by knowledge of the underlying genetic and biochemical defects responsible for the behavior. CHRNA4 is involved in ADHD pathogenesis and the development of regular smoking behavior and will be a target for drug discovery efforts. The associated SNPs described here are useful in risk assessment of smoking behavior and likely also for risk of use of other stimulant medication.

Polymorphism in CHRNA4 has also been associated with inattentive ADHD. The present findings are also are consistent with the existence of a biological basis for the frequent observation that inattentive forms of attention deficit/hyperactivity disorder (ADHD) commonly co-occur with regular smoking behavior. Clustering of genes for complex traits is not unusual. For example, a cluster of genes for murine systemic lupus erythematosus (SLE) exists on chromosome 1q, and there are likely to be two Crohn's disease loci within approximately 20cM on chromosome 16q. As with any complex disease study it will be necessary to examine association of these variants with other cohorts, as well as with other related diseases or behaviors such as use or abuse of other stimulant medications.

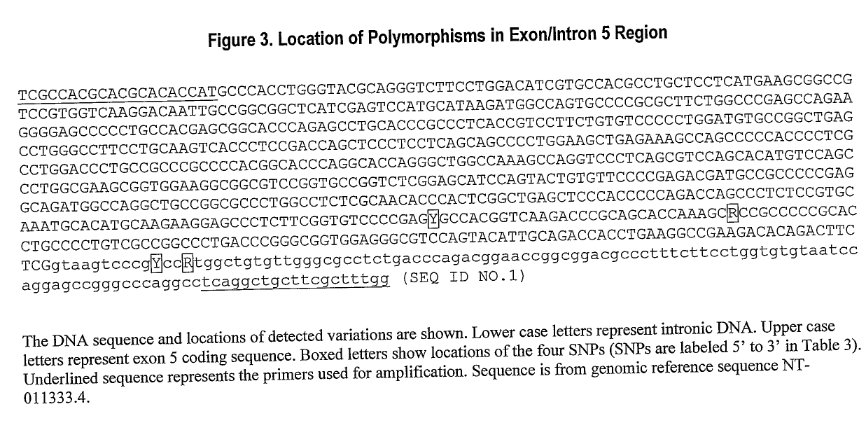

Accordingly, in one aspect the present invention provides methods and related materials for determining risk in an individual of developing regular smoking behavior. An exemplary embodiment of the method includes, in a biological sample from the subject, analyzing a polynucleotide sequence to detect the presence or absence of an allelic variant of a polymorphic region of exon/intron 5 region of CHRNA4. The polymorphic region comprises the sequence of SEQ ID NO: 1, as shown in FIG. 3. The allelic variant of the polymorphic region includes at least one, or both, of the SNP's identified herein as snp 1 T and snp 2 A. In order to detect the presence or absence of the allelic variant, the method encompasses using a method selected from the group consisting of: allele specific hybridization, primer specific extension, oligonucleotide ligation assay, restriction enzyme site analysis and single-stranded conformation polymorphism analysis.

A test sample of nucleic acid suitable for diagnostic testing according to the methods described herein, is conveniently a sample of blood, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique, such as PCR, before use in the analysis of the polymorphic region of CHRNA4.

Methods used for diagnosis are, for example, those in which the sequence is determined by a method such as amplification refractory mutation system and restriction fragment length polymorphism (RFLP). It will be apparent to the person skilled in the art that there are a large number of analytical procedures that may be used to detect the presence or absence of variant nucleotides at one or more of the SNP positions described herein with respect to CHRNA4. In general, the detection of allelic variation requires a mutation discrimination technique, optionally an amplification reaction and a signal generation system.

Mutation detection techniques can be based, for example, on the PCR. Exemplary techniques, without limitation, include the following: general techniques such as DNA sequencing, sequencing by hybridization; scanning techniques such as PJT*, SSCP, DOGE, TGGE, Cleavase, Heteroduplex analysis, CMC, Enzymatic mismatch cleavage; hybridization based solid phase hybridization such as dot blots, MASDA, reverse dot blots, oligonucleotide arrays (DNA Chips); solution phase hybridization such as Taqman™ (U.S. Pat. Nos. 5,210,015 & 5,487,972, Hoffmann-La Roche); molecular beacons such as that described by Tyagi et al (1996), Nature Biotechnology 14,303 and WO 95/13399 (Public Health Inst., New York); extension based techniques such as ARMS™, ALEX™ (European Patent No. EP 332435 B1, Zeneca Limited) and COPS (Gibbs et al, Nucleic Acids Research, 17, 2347 (1989); incorporation based techniques such as mini-sequencing, and APEX; restriction enzyme based, such as RFLP, restriction site generating PCR; ligation based such as OLA; and other techniques such as invader assays. These techniques will often be used in combination with a number of signal generation systems.

Signal detection techniques include, for example, fluorescence-based techniques such as FRET, fluorescence quenching, fluorescence polarization (United Kingdom Patent No. 2228998, Zeneca Limited); calorimetric assays such as hybridization protection assay; mass spectrometry, and other signal detection techniques such as chemiluminescence, electrochemiluminescence, Raman spectroscopy and radioactivity signal detection.

Many current methods for the detection and amplification of allelic variation can be found in recent reviews, such as that by Nollau et al., Clin. Chem. 43, 1114-1120 (1997), and also in standard textbooks, such as, for example “Laboratory Protocols for Mutation Detection”, Ed. by U. Landegren, Oxford University Press, (1996), and “PCR”, 2nd Ed., Newton & Graham, BIOS Scientific Publishers Limited, (1997).

In another embodiment of the invention, a method for determining whether a subject is at risk for developing regular smoking behavior, includes, in a biological sample from the subject, analyzing a polynucleotide sequence to detect the presence or absence of at least one DNA marker in the exon/intron 5 region of CHRNA4, wherein the DNA marker is associated with increased risk of developing regular smoking behavior. The DNA marker is one of sup 1 T or snp 2 A, or both alleles.

In another embodiment of the invention, a method for detecting the presence or absence in a subject of at least one allelic variant that is associated with increased risk of developing regular smoking behavior comprises detecting the presence or absence of at least one SNP in exon/intron 5 region of CHRNA4, wherein the presence of at least one SNP is associated with increased risk of developing regular smoking behavior. Again, the SNP is selected from the snp 1 T and snp 2 A alleles, and the allelic variant can include both SNP's.

In another embodiment of the invention, there is provided a method for detecting in a subject a predisposition to developing regular smoking behavior, comprising, on a CHRNA4 gene obtained from the subject, detecting the presence or absence of an allelic variant of the polymorphic region of exon/intron 5 that is associated with increased risk of developing regular smoking behavior as described herein, wherein the polymorphic region comprises SEQ ID NO: 1, and wherein the presence of the allelic variant in the subject is indicative of a predisposition to developing regular smoking behavior in the subject as compared to a subject in which the allelic variant is not present. Again, the SNP is selected from the snp 1 T and snp 2 A alleles, and the allelic variant can include both SNP's.

The materials also encompass a primer or probe that specifically hybridizes adjacent to or at or at a polymorphic region of CHRNA4 gene having the nucleotide sequence of SEQ ID NO: 1, wherein allelic variants of the polymorphic region are associated with increased risk of developing regular smoking. The allelic variant includes one or both of the snp1T and snp2A alleles. A diagnostic primer is defined as a nucleic acid and an allele specific primer that is used, generally together with a constant primer, in an amplification reaction such as a PCR reaction, which provides the discrimination between alleles through selective amplification of one allele at a particular sequence position, such as used for ARMS™ assays. The diagnostic primer is preferably 10-50 nucleotides. Provided are diagnostic primers or probes including combinations of sequences encompassing the allelic variations identified here as associating with increased risk of developing regular smoking behavior The primers may be manufactured using any convenient method of synthesis. Examples of such methods may be found in standard textbooks, for example “Protocols for Oligonucleotides and Analogues; Synthesis and Properties,” Methods in Molecular Biology Series, Vol. 20; Ed. Sudhir Agrawal, Humana ISBN: 0-89603-247-7; 1993; 1st Edition. If required the primer(s) may be labelled with signal-generating materials to facilitate detection.

In another embodiment of the invention, there is provided a kit for indicating whether a subject has an increased risk of developing regular smoking behavior, the kit comprising: at least one probe or primer that specifically hybridizes adjacent to or at a polymorphic region of CHRNA4 comprising SEQ ID NO: 1, wherein allelic variants of the polymorphic region are associated with increased risk of developing regular smoking; and instructions for use of the kit for indicating whether the subject has increased risk of developing regular smoking behavior. In one embodiment of the kit, the polymorphic region comprises an allelic variant comprising a SNP selected from the snp1T and snp2A alleles. In another embodiment of the kit, the polymorphic region comprises an allelic variant comprising two SNPs consisting of the snp1T and snp2A alleles. The kit instructions, for example, may identify the two SNPs in the polymorphic region of CHRNA4, and may further describe how to detect the presence or absence of any such variants within a biological sample taken from a subject. The kits may comprise appropriate packaging and may further comprise appropriate buffer(s) and polymerase(s) such as thermostable polymerases, for example Taq polymerase.

In another aspect, microarrays useful for diagnosis are provided which include, for example, a nucleic acid having a sequence of a polymorphic region of CHRNA4 comprising SEQ ID NO: 1, and that is associated with increased risk of developing regular smoking behavior. In one embodiment of the microarray, the polymorphic region comprises an allelic variant comprising a SNP selected from the snp1T and snp2A alleles. In another embodiment of the microarray, the polymorphic region comprises an allelic variant comprising two SNPs consisting of the snp1T and snp2A alleles.

Individuals who carry the particular allelic variants of CHRNA4, may exhibit differences in their ability to produce or regulate the subject receptor protein subunits or isoforms under different physiological conditions, and will thus display altered abilities to react to different factors that play a role in the development of smoking behavior. In addition, differences in protein expression and regulation arising as a result of allelic variation may have a direct effect on the response of an individual to drug therapy. The polymorphisms described herein may therefore have the greatest effect on the efficacy of drugs designed to modulate the activity of CHRNA4. The diagnostic methods of the invention may therefore be useful both to predict the clinical response to such drug agents and to determine therapeutic dose.

In a further aspect, the diagnostic methods of the invention are used in the development of new drug therapies that selectively target one or more allelic variants of CHRNA4. Polymorphisms, SNP's, have been extremely useful for mapping the human genome and continue to help elucidate the genetic component of diseases. While lists of numerous, previously identified polymorphisms are generally available, for example in online databases, most are not yet known to have an association with any particular protein, function or disease. However, the present results demonstrate the clear association of the snp 1 T and snp 2 A alleles with increased risk of developing regular smoking behavior.

The particular allelic variants of CHRNA4 that have been identified by the inventors as being associated with increased risk of developing regular smoking behavior, can be used to screen for new pharmaceutical compounds that preferentially act on CHRNA4 protein products and associated pathways in order to develop more effective treatments for regular smoking behavior and nicotine dependence. The field of pharmacogenetics approaches treatment of disease using such genetic knowledge to diagnose disease and identify patients most amenable to treatment using particular pharmaceutical agents. Known pharmacogenetic techniques can be used in pharmaceutical research to assist the drug selection process. Drugs may be designed to regulate the biological activity of variants implicated in the behavioral development process whilst minimizing effects on other variants. References that provide background details on pharmacogenetics and other uses of polymorphism detection include, for example, Linder et al., Clinical Chemistry 43, 254 (1997); Marshall, Nature Biotechnology 15, 1249 (1997); International Patent Application WO 97/40462, Spectra Biomedical; and Schafer et al., Nature Biotechnology 16, 33 (1998). General molecular biology procedures can be followed from any of the methods described in “Molecular Cloning—A Laboratory Manual” Second Edition, Sambrook, Fritsch and Maniatis (Cold Spring Harbor Laboratory, 1989).

In order to further illustrate the invention, the following specific laboratory examples are described, although it will be understood that the invention is not limited to these specific examples or the details described therein.

### EXAMPLES

### Example 1

### Population-Based Study of Association Between CHRNA4 Polymorphism Status and Regular Smoking

In order to attempt to link the association of a CHRNA4 polymorphism with inattentive ADHD and the previously described associations of smoking with attention problems, predictors of regular smoking were analyzed in a population-based sample of 1430. Risk for regular smoking was then analyzed for an association with CHRNA4 polymorphism status. As shown in Table 1, the sample consisted of 831 males and 509 females aged 7 to 17 years. The majority of individuals receiving a diagnosis of ADHD by either DSM-IV or latent class criteria (the inattentive and combined subtypes) were males but there was no difference in age or ethnicity by subtype. Consistent with an earlier report (Todd R D, Sitdhiraksa N, Reich W, Ji T H-C, Joyner C A, Neuman R J, Heath A C (2002), Discrimination of DSM-IV and latent class attention deficit/hyperactivity disorder subtypes by educational and cognitive performance in a population based sample of child and adolescent twins. Journal of the American Academy of Child and Adolescent Psychiatry 41: 820-828), verbal IQ scores were somewhat lower for combined subtype ADHD classes. Similarly, parent reported use of stimulant medication was higher for the combined subtypes than for inattentive or no ADHD.

As part of the standardized diagnostic interview process using the MAGIC interview (see Todd R D, Joyner C A, Heath A C, Neuman R J, Reich W (2003), Reliability and stability of a semistructured DSM-IV interview designed for family studies. Journal of the American Academy of Child & Adolescent Psychiatry. 42: 1460-8), questions regarding smoking by self or friends, alcohol and drug use were systematically asked of all participants. Full DSM-IV diagnoses of alcohol abuse and dependence could be made though full criteria for nicotine dependence was not queried (Table 2). Not correcting for covariates, there was an increased frequency of having had a drink or having smoked marijuana in males versus females as well as a higher rate of an alcohol abuse diagnoses. Regular smoking was strongly correlated with having friends who smoke (p<0.0001). Individuals with any DSM-IV ADHD diagnosis also had a higher rate of ever having smoked regularly or having smoked marijuana.

When analyzed by latent class subtype, there were significant increases in having ever regularly smoked or ever smoked marijuana, as well as ever having had a drink or ever being drunk in the inattentive ADHD class compared to the no symptoms class. There were no significant increased frequencies for latent class defined combined subtype.

### Example 2

### Multivariate Logistic Regression Analysis of Association Between CHRNA4 Polymorphism Status and Regular Smoking

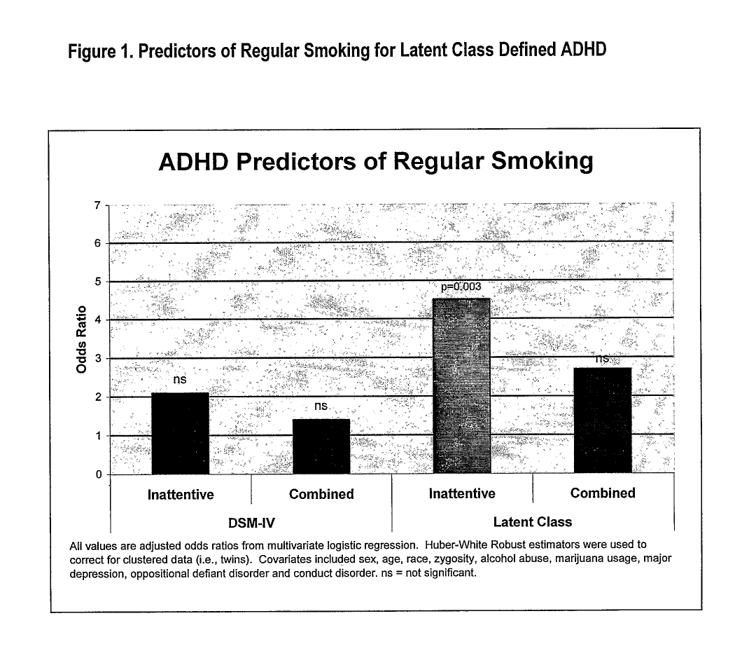

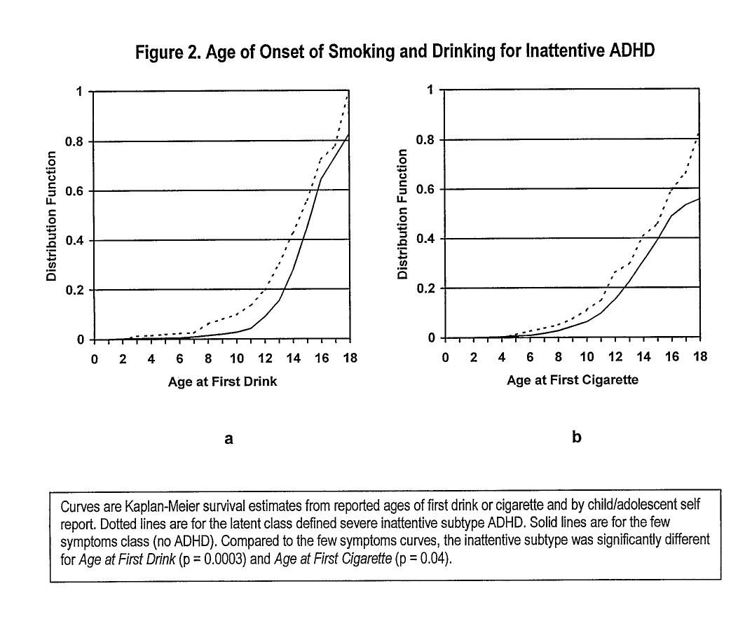

Since smoking is associated with a variety of possible confounding individual characteristics, the magnitude of ADHD predictors of regular smoking was estimated using multivariate logistic regression. Covariates included sex, age, race, zygocity, alcohol abuse, marijuana usage, major depression, oppositional defiant disorder, conduct disorder and ADHD subtype. Due to its high correlation with self-reported smoking, friend's smoking could not be included as a separate covariate. Since these were twin-based data, Huber-White Robust estimators were used to estimate confidence intervals. FIG. 1 shows that though the adjusted odds ratios for prediction of regular smoking were slightly elevated in DSM-IV ADHD subtypes, these were not significant. A significant odds ratio was only found for the inattentive latent class subtype (OR=4.5, 95% CI 1.7-11.8, p=0.003). For this subtype, smoking status was also predicted by age (p=0.003), history of alcohol abuse (p=0.03), and history of marijuana smoking (p<0.001), but not by sex, race (African-American or not), zygosity, major depression, oppositional defiant disorder or conduct disorder (data not shown). FIG. 2 shows that Kaplan-Meir Survival estimates for age of first cigarette or first drink were not significantly less for inattentive ADHD but lifetime risks were higher. The estimated risk of trying a cigarette by age 18 years was 82% for inattentive ADHD versus 55% for non-ADHD individuals.

### Example 3

### Multivariate Logistic Regression Analysis of Association Between CHRNA4 Polymorphism Status and Regular Smoking

Given that smoking was predicted by latent class ADHD subtype, CHRNA4 genotyping data for the inattentive and combined ADHD subtypes was analyzed to test for association with the presence or absence of smoking. All snp genotype assignments from Todd et al. (Todd R D, Joyner C A, Heath A C, Neuman R J, Reich W (2003), Reliability and stability of a semistructured DSM-IV interview designed for family studies. Journal of the American Academy of Child & Adolescent Psychiatry. 42: 1460-8) were confirmed by genomic DNA sequencing. Analyses were restricted to those individuals who had ever smoked, reducing the sample size from 195 to 76 individuals. As shown in Table 3, there were significant associations of two exon/intron 5 snps with progression to regular smoking when all ADHD individuals were grouped together (OR=2.1, p=0.03 for each). When analyzed separately by combined and inattentive latent class subtypes, the results were statistically significant only for the inattentive subtype (OR=2.7, p=0.02 for each). As shown in Table 3, there was an increase of snp 1 T alleles and an increase of snp 2 A alleles in the regular smoking group. Similar results were found for haplotype analysis since these 2 snps were in complete linkage disequilibrium. Post hoc analyses of other snps and haplotypes described in Todd et al. (Todd et al. (2003), Journal of the American Academy of Child & Adolescent Psychiatry. 42: 1460-8), were not significant.

### OTHER EMBODIMENTS

When introducing elements of the present invention or the preferred embodiments thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

TABLE 1
Sample Characteristics

| | | Percent | Average Age | Percentage of | Verbal IQ^{b} | Percent Used |
|---|---|---|---|---|---|---|
| Group | Number | Male | (years ± std) | African-American^{a} | (mean ± SD) | ADHD Medication^{c} |
| Total | 1340 | 62.0 | 14.0 ± 2.6 | 13.7 | 8.9 ± 3.0 | 10.1 |
| Males | 831 | 100.0 | 14.2 ± 2.6 | 13.4 | 8.9 ± 3.0 | 13.7 |
| Females | 509 | 0.0 | 13.6 ± 2.6 | 14.2 | 8.8 ± 3.0 | 4.1 |
| DSM-IV - No ADHD | 1164 | 58.5 | 14.0 ± 2.6 | 13.8 | 9.0 ± 2.9 | 6.8 |
| DSM-IV ADHD | 176 | 85.2 | 13.6 ± 2.6 | 12.5 | 7.8 ± 3.3 | 31.8 |
| DSM-IV Inattentive | 96 | 84.4 | 13.9 ± 2.7 | 14.6 | 8.5 ± 3.3 | 29.2 |
| DSM-IV Combined | 65 | 87.7 | 13.3 ± 2.4 | 9.2 | 6.8 ± 3.3 | 32.3 |
| Latent Class - Few | 743 | 52.4 | 14.1 ± 2.6 | 11.7 | 9.4 ± 2.8 | 2.6 |
| Latent Class - Inattentive | 83 | 83.1 | 14.4 ± 2.5 | 12.1 | 8.4 ± 2.9 | 25.3 |
| Latent Class -Combined | 100 | 77.0 | 13.1 ± 2.4 | 14.0 | 6.9 ± 3.4 | 32.0 |

LC = latent class defined ADHD subtype.
^{a}= Parent reported ethnic/racial group. Remainder were 84% European-American with less than 3% other ethnic/racial groups.
^{b}= Assessed using the Vocabulary subscale of Weschler Intelligence Scale for Children (WISC-III)
^{c}= Parent reported lifetime child use of medication for ADHD problems. This was a methylphenidate-based product 77.2% of the time.

TABLE 2
Endorsement Rates of Selected Questions and Diagnoses by Sex and ADHD Status

| | | | | | | | | | LCA | | LCA | |
|---|---|---|---|---|---|---|---|---|---|---|---|---|
| | Total | | Males | | Females | | DSM ADHD | | Combined ADHD | | Inattentive ADHD | |
| | (n = 1335) | | (n = 829) | | (n = 506) | | (n = 174) | | (n = 98) | | (n = 83) | |
| Question | Yes | % | Yes | % | Yes | % | Yes | % | Yes | % | Yes | % |
| 1) Ever smoked cigarettes? | 463 | 34.7 | 316 | 38.1 | 147 | 29.0** | 69 | 39.4 | 35 | 35.7 | 35 | 42.2 |
| 2) Ever smoked regularly?^{a} | 192 | 41.5 | 137 | 43.4 | 55 | 37.4 | 37 | 53.6* | 18 | 51.4 | 26 | 74.3*** |
| 3) Ever smoked marijuana? | 225 | 16.9 | 168 | 20.3 | 57 | 11.3*** | 44 | 25.3** | 21 | 21.4 | 28 | 33.7*** |
| 4) Smoked marijuana more than twice?^{b} | 164 | 72.9 | 122 | 72.6 | 42 | 73.7 | 29 | 65.9 | 16 | 76.2 | 19 | 67.9 |
| 5) Ever had a drink? | 500 | 37.5 | 346 | 41.7 | 154 | 30.4*** | 63 | 36.2 | 32 | 32.7 | 43 | 51.8** |
| 6) Ever been drunk?^{c} | 191 | 38.2 | 140 | 40.5 | 51 | 33.1 | 27 | 42.9 | 13 | 40.6 | 23 | 53.5** |
| 7) Alcohol abuse diagnosis? | 127 | 9.5 | 92 | 11.1 | 35 | 6.8* | 19 | 10.8 | 8 | 8.2 | 12 | 14.1 |
| 8) Alcohol dependence diagnosis? | 0 | 0 | 0 | 0 | 0 | 0 | 0 | 0 | 0 | 0 | 0 | 0 |

All statistical comparisons are Kruskal-Wallis tests for males versus females, DSM-IV ADHD versus no ADHD or latent class few symptoms class versus latent class severe combined or severe inattentive latent class ADHD subtypes.
p values of Kruskal-Wallis One Way Comparisons -
***p ≦ 0.001,
**p ≦ 0.01,
*p ≦ 0.05.
^{a}of those who had ever smoked a cigarette
^{b}of those who had ever smoked marijuana
^{c}of those who had ever had a drink

TABLE 3
CHRNA4 Exon/Intron 5 Allele Frequencies

| | |
|---|---|
| Inattentive | Combined |

| snp 1 | | T | C | T | C |
|---|---|---|---|---|---|
| Regular | No | 13 | 17 | 18 | 30 |
| Smoker | Yes | 27 | 13 | 13 | 17 |
| OR | | 2.7 (1.1-6.5) | | 1.3 (0.5-3.4) | |
| snp 2 | | A | G | A | G |
| Regular | No | 13 | 17 | 18 | 30 |
| Smoker | Yes | 27 | 13 | 13 | 17 |
| OR | | 2.7 (1.1-6.5) | | 1.3 (0.5-3.4) | |
| snp 3 | | G | A | G | A |
| Regular | No | 30 | 0 | 48 | 0 |
| Smoker | Yes | 37 | 3 | 29 | 1 |
| OR | | NA | | NA | |
| snp 4 | | A | G | A | G |
| Regular | No | 24 | 6 | 36 | 12 |
| Smoker | Yes | 33 | 7 | 22 | 8 |

| | | |
|---|---|---|
| OR | 0.8 (0.3-2.7) | 1.1 (0.4-2.7) |

### REFERENCES

1. Lightwood J (2003), The economics of smoking and cardiovascular disease. Progress in Cardiovascular Diseases. 46: 39-78
2. Kiiskinen U, Vartiainen E, Puska P, Pekurinen M (2002), Smoking-related costs among 25 to 59 year-old males in a 19-year individual follow-up. European Journal of Public Health. 12: 145-51
3. Max W (2001), The financial impact of smoking on health-related costs: a review of the literature. American Journal of Health Promotion. 15: 321-31
4. Tyndale R F (2003), Genetics of alcohol and tobacco use in humans. Annals of Medicine. 35: 94-121
5. Sullivan P F, Kendler K S (1999), The genetic epidemiology of smoking. Nicotine & Tobacco Research. 1: S51-7; discussion S69-70
6. Hogg R C, Raggenbass M, Bertrand D (2003), Nicotinic acetylcholine receptors: from structure to brain function. Reviews of Physiology Biochemistry & Pharmacology. 147: 1-46
7. Champtiaux N, Changeux JP (2004), Knockout and knockin mice to investigate the role of nicotinic receptors in the central nervous system. Progress in Brain Research. 145: 235-51
8. Alkondon M, Albuquerque E X (2004), The nicotinic acetylcholine receptor subtypes and their function in the hippocampus and cerebral cortex. Progress in Brain Research. 145: 109-20
9. Zhou F M, Wilson C, Dani J A (2003), Muscarinic and nicotinic cholinergic mechanisms in the mesostriatal dopamine systems. Neuroscientist. 9: 23-36
10. Zhou F M, Liang Y, Dani J A (2001), Endogenous nicotinic cholinergic activity regulates dopamine release in the striatum. Nature Neuroscience. 4: 1224-9
11. Owens J C, Balogh S A, McClure-Begley T D, Butt C M, Labarca C, Lester H A, Picciotto M R, Wehner J M, Collins A C (2003), Alpha4beta2* Nicotinic Acetylcholine Receptors Modulate the Effects of Ethanol and Nicotine on the Acoustic Startle Response. Alcoholism: Clinical and Experimental Research 27: 1867-1875
12. Cohen C, Bergis O E, Galli F, Lochead A W, Jegham S, Biton B, Leonardon J, Avenet P, Sgard F, Besnard F, Graham D, Coste A, Oblin A, Curet O, Voltz C, Gardes A, Caille D, Perrault G, George P, Soubrie P, Scatton B (2003), SSR591813, a novel selective and partial alpha4beta2 nicotinic receptor agonist with potential as an aid to smoking cessation. Journal of Pharmacology & Experimental Therapeutics. 306: 407-20
13. Ueno K, Togashi H, Matsumoto M, Ohashi S, Saito H, Yoshioka M (2002), Alpha4beta2 nicotinic acetylcholine receptor activation ameliorates impairment of spontaneous alternation behavior in stroke-prone spontaneously hypertensive rats, an animal model of attention deficit hyperactivity disorder. Journal of Pharmacology & Experimental Therapeutics. 302: 95-100
14. Silverman M A, Neale M C, Sullivan P F, Harris-Kerr C, Wormley B, Sadek H, Ma Y, Kendler K S, Straub R E (2000), Haplotypes of four novel single nucleotide polymorphisms in the nicotinic acetylcholine receptor beta2-subunit (CHRNB2) gene show no association with smoking initiation or nicotine dependence. American Journal of Medical Genetics 96: 646-53
15. Lueders K K, Hu S, McHugh L, Myakishev M V, Sirota L A, Hamer D H (2002), Genetic and functional analysis of single nucleotide polymorphisms in the beta2-neuronal nicotinic acetylcholine receptor gene (CHRNB2). Nicotine & Tobacco Research. 4: 115-2
16. Burke J D, Loeber R, Lahey B B (2001), Which aspects of ADHD are associated with tobacco use in early adolescence? Journal of Child Psychology and Psychiatry and Allied Disciplines 42: 493-50
17. Barkley R A, Fischer M, Edelbrock C S, Smallish L (1990), The adolescent outcome of hyperactive children diagnosed by research criteria: I. An 8-year prospective follow-up study. Journal of the American Academy of Child and Adolescent Psychiatry 29: 546-557
18. Hartsough C S, Lambert N M (1987), Pattern and progression of drug use among hyperactives and controls: A prospective short-term longitudinal study. Journal of Child Psychology and Psychiatry and Allied Disciplines 28: 543-553
19. Lambert N M, Hartsough C S (1998), Prospective study of tobacco smoking and substance dependencies among samples of ADHD and non-ADHD participants. Journal of Learning Disabilities. 31: 533-44
20. Milberger S, Biederman J, Faraone S V, Chen L, Jones J (1997), ADHD is associated with early initiation of cigarette smoking in children and adolescents. Journal of the American Academy of Child and Adolescent Psychiatry 36: 37-44
21. Tercyak K P, Lerman C, Audrain J (2002), Association of attention-deficit/hyperactivity disorder symptoms with levels of cigarette smoking in a community sample of adolescents. Journal of the American Academy of Child and Adolescent Psychiatry 41: 799-805
22. Lerman C, Audrain J, Tercyak K, Hawk L W, Jr., Bush A, Crystal-Mansour S, Rose C, Niaura R, Epstein L H (2001), Attention-Deficit Hyperactivity Disorder (ADHD) symptoms and smoking patterns among participants in a smoking-cessation program. Nicotine & Tobacco Research. 3: 353-9
23. Conners C K, Levin E D, Sparrow E, Hinton S C, Erhardt D, Meck W H, Rose J E, March J (1996), Nicotine and attention in adult attention deficit hyperactivity disorder (ADHD). Psychopharmacology Bulletin 32: 67-73
24. Levin E D, Conners C K, Sparrow E, Hinton S C, Erhardt D, Meck W H, Rose J E, March J (1996), Nicotine effects on adults with attention-deficit/hyperactivity disorder. Psychopharmacology (Berl) 123: 55-63
25. Levin E D, Conners C K, Silva D, Hinton S C, Meck W H, March J, Rose J E (1998), Transdernal nicotine effects on attention. Psychopharmacology (Berl) 140: 135-141
26. Levin E D, Conners C K, Silva D, Canu W, March J (2001), Effects of chronic nicotine and methylphenidate in adults with attention deficit/hyperactivity disorder. Experimental and Clinical Psychopharmacology 9: 83-90
27. Shytle R D, Silver A A, Wilkinson B J, Sanberg P R (2002), A pilot controlled trial of transdermal nicotine in the treatment of attention deficit hyperactivity disorder. World Journal of Biological Psychiatry 3: 150-155
28. Todd R D, Lobos E A, Sun L W, Neuman R J (2003), Mutational analysis of the nicotinic acetylcholine receptor alpha 4 subunit gene in attention deficit/hyperactivity disorder: evidence for association of an intronic polymorphism with attention problems. Molecular Psychiatry 8: 103-108
29. Todd R D, Rasmussen E R, Neuman R J, Reich W, Hudziak J J, Bucholz K K, Madden P A F, Heath A C (2001), Familiarity and heritability of subtypes of attention deficit hyperactivity disorder in a population sample of adolescent female twins. American Journal of Psychiatry 158: 1891-1898
30. Rasmussen E R, Neuman R J, Heath A C, Levy F, Hay D A, Todd R D (2004), Familial clustering of latent class and DSM-IV defined attention-deficit/hyperactivity disorder subtypes. Journal of Child Psychology & Psychiatry & Allied Disciplines. 45: 589-598
31. Todd R D, Sitdhiraksa N, Reich W, Ti T H-C, Joyner C A, Neuman R J, Heath A C (2002), Discrimination of DSM-IV and latent class attention deficit/hyperactivity disorder subtypes by educational and cognitive performance in a population based sample of child and adolescent twins. Journal of the American Academy of Child and Adolescent Psychiatry 41: 820-828
32. Todd R D, Joyner C A, Heath A C, Neuman R J, Reich W (2003), Reliability and stability of a semistructured DSM-IV interview designed for family studies. Journal of the American Academy of Child & Adolescent Psychiatry. 42: 1460-8
33. Reich W (2000), Diagnostic interview for children and adolescents (DICA). Journal of the American Academy of Child and Adolescent Psychiatry 39: 59-66
34. Rasmussen E R, Neuman R J, Heath A C, Levy F, Hay D A, Todd RD (2002), Replication of the latent class structure of Attention-Deficit/Hyperactivity Disorder (ADHD) subtypes in a sample of Australian twins. Journal of Child Psychology and Psychiatry and Allied Disciplines 43: 1018-28
35. Rasmussen E R, Neuman R J, Heath A C, Levy F, Hay D A, Todd R D (2004), Familial clustering of latent class and DSM-IV defined attention-deficit/hyperactivity disorder subtypes. Journal of Child Psychology and Psychiatry and Allied Disciplines 45: 589-98
36. Hudziak J J, Rudiger L P, Neale M C, Heath A C, Todd R D (2000), A twin study of inattentive, aggressive, and anxious/depressed behaviors. Journal of the American Academy of Child and Adolescent Psychiatry 39: 469-76
37. Todd R D, Sitdhiraksa N, Reich W, Ji T H, Joyner C A, Heath A C, Neuman R J (2002), Discrimination of DSM-IV and latent class attention-deficit/hyperactivity disorder subtypes by educational and cognitive performance in a population-based sample of child and adolescent twins. Journal of the American Academy of Child and Adolescent Psychiatry 41: 820-8
38. SAS Institute Inc., Version 8.2. Cary, N.C., USA
39. Stata Statistical Software: Release 7.0. College Station, TX: Stata Corporation

1. A method for determining risk of developing regular smoking behavior, said method comprising: in a biological sample from the subject, analyzing a polynucleotide sequence to detect the presence or absence of (i) an allelic variant of a polymorphic region of exon/intron 5 region of CHRNA4 or (ii) at least one DNA marker in exon/intron 5 region of CHRNA4, wherein the DNA marker is associated with increased risk of developing regular smoking behavior.

2. A method in accordance with claim 1 wherein the polymorphic region comprises the sequence of SEQ ID NO: 1.

3. A method in accordance with claim 1 wherein the allelic variant or the at least one DNA marker comprises at least one single nucleotide polymorphism (SNP).

4. A method in accordance with claim 3 wherein the at least one single nucleotide polymorphism is selected from the group consisting of snp 1 T and snp 2 A alleles.

5. A method in accordance with claim 4 wherein the allelic variant or the at least one DNA marker comprises two single nucleotide polymorphisms (SNPs) consisting of snp 1 T and snp 2 A alleles.

6-8. (canceled)

9. A method in accordance with claim 3, wherein the presence of at least one SNP is associated with increased risk of developing regular smoking behavior.

10. A method in accordance with claim 3 wherein detecting the presence or absence the allelic variant or the at least one DNA marker comprises detecting the presence or absence of at least one SNP selected from the group consisting of snp 1 T and snp 2 A alleles.

11. A method in accordance with claim 10 wherein detecting the presence or absence of at least one SNP comprises detecting the presence or absence of two SNPs consisting of snp 1 T and snp 2 A alleles.

12. A method in accordance with claim 1 wherein the sample comprises a CHRNA4 gene obtained from the subject, the polymorphic region comprises SEQ ID NO: 1, and the presence of the allelic variant in the subject is indicative of a predisposition to developing regular smoking behavior in the subject as compared to a subject in which the allelic variant is not present.

13-14. (canceled)

15. A method in accordance with claim 1 wherein detecting the presence or absence of the allelic variant or the at least one DNA marker comprises a method selected from the group consisting of: allele specific hybridization, primer specific extension, oligonucleotide ligation assay, restriction enzyme site analysis and single-stranded conformation polymorphism analysis.

16-18. (canceled)

19. A kit for indicating whether a subject has an increased risk of developing regular smoking behavior, comprising: at least one probe or primer that specifically hybridizes adjacent to or at a polymorphic region of CHRNA4 comprising SEQ ID NO: 1 wherein allelic variants of the polymorphic region are associated with increased risk of developing regular smoking.

20. A kit in accordance with claim 19 wherein the polymorphic region comprises an allelic variant comprising a SNP selected from the group consisting of snpIT and snp2A.

21. A kit in accordance with claim 19 wherein the polymorphic region comprises an allelic variant comprising two SNPs consisting of snpIT and snp2A.

22. A microarray, comprising a nucleic acid having a sequence of a polymorphic region of CHRNA4 comprising SEQ ID NO: 1, and that is associated with increased risk of developing regular smoking behavior.

23. A microarray in accordance with claim 22 wherein the polymorphic region comprises an allelic variant comprising a SNP selected from the group consisting of snpIT and snp2A.

24. A microarray in accordance with claim 22 wherein the polymorphic region comprises an allelic variant comprising two SNPs consisting of snpIT and snp2A.


SEQUENCE LISTING (1 sequence)

SEQ ID NO: 1 - DNA, 877 bp, Homo sapiens
tcgccacgcacgcacaccatgcccacctgggtacgcagggtcttcctggacatcgtgccacgcctgctcctcatgaagcggccgtccgtggtcaaggacaattgccggcggctcatcgagtccatgcataagatggccagtgccccgcgcttctggcccgagccagaaggggagccccctgccacgagcggcacccagagcctgcacccgccctcaccgtccttctgtgtccccctggatgtgccggctgagcctgggccttcctgcaagtcaccctccgaccagctccctcctcagcagcccctggaagctgagaaagccagcccccacccctcgcctggaccctgccgcccgccccacggcacccaggcaccagggctggccaaagccaggtccctcagcgtccagcacatgtccagccctggcgaagcggtggaaggcggcgtccggtgccggtctcggagcatccagtactgtgttccccgagacgatgccgcccccgaggcagatggccaggctgccggcgccctggcctctcgcaacacccactcggctgagctcccacccccagaccagccctctccgtgcaaatgcacatgcaagaaggagccctcttcggtgtccccgagygccacggtcaagacccgcagcaccaaagcrccgcccccgcacctgcccctgtcgccggccctgacccgggcggtggagggcgtccagtacattgcagaccacctgaaggccgaagacacagacttctcggtaagtcccgyccrtggctgtgttgggcgcctctgacccagacggaaccggcggacgccctttcttcctggtgtgtaatccaggagccgggcccaggcctcaggctgcttcgctttgg